(19) United States
(12) Patent Application Publication
(10) Pub. No.: US 2006/0204469 A1
(43) Pub. Date: Sep. 14, 2006

(54) Stable mixed emulsions

(76) Inventors: Eric Spengler, Ridgefield, CT (US); Teresa Petraia, Norwalk, CT (US)

Correspondence Address: FITZPATRICK CELLA HARPER & SCINTO, 30 ROCKEFELLER PLAZA, NEW YORK, NY, 10112, US

(21) Appl. No.: 11/369,831

(22) Filed: Mar. 8, 2006

Related U.S. Application Data
(60) Provisional application No. 60/659,410, filed on Mar. 9, 2005.

(51) Int. Cl. A61K 8/36; A61K 8/89

(52) U.S. Cl. 424/70.31; 424/401; 977/926

(57) ABSTRACT

Stable mixed emulsion systems containing at least two discrete and distinctly different dispersed particle size ranges, i.e., both nanoemulsion and macroemulsion semisolid dispersions, as well as a method of making the same, are disclosed. The stable mixed emulsions may be used for cosmetic and dermatological applications and provide unique benefits.

This application claims the benefit of U.S. Provisional Patent Application No. 60/659,410, filed Mar. 9, 2005.

### BACKGROUND OF THE INVENTION

1. Field of the Invention

This invention relates to stable mixed emulsions—systems containing both nanoemulsion semisolid dispersions and macroemulsion semisolid dispersions, as well as to methods of making the same and to skin care compositions comprising the same.

2. Related Background Art

The personal care and pharmaceutical industry constantly searches for new technologies to deliver better performance to target organs such as skin. To that end, nanoemulsion technology is gaining interest due to its reported benefits. For example, Tadros et al. (“Stabilizing Nanodispersions in Personal Care Applications”, Cosmetics & Toiletries, vol. 119, no. 8, pp. 41-46 (2004)) claim the following benefits for nanoemulsions:
efficient delivery of skin care actives
due to small particle size, can penetrate through rough skin surface and enhance penetration of actives
can be prepared using reasonable surfactant (emulsifier) concentrations—Microemulsions, another form of submicron emulsions, typically require very high levels of surfactants, sometimes to a level which can cause safety concerns. Nanoemulsions yield kinetic stability while microemulsions yield both thermodynamic and kinetic stability
small droplet size allows for uniform deposition on substrates
can be used for delivery of fragrances, enabling preparation of perfumes that are alcohol free
may be applied as a substitute for liposomes and vesicles which are much less stable and (much more) expensive to produce.

There are three known basic processes for creating a nanoemulsion. The first is the Phase Inversion Temperature (PIT) method. See, e.g., U.S. Pat. Nos. 6,537,562 and 6,716,443. The second method is high pressure homogenization. See, e.g., U.S. Pat. No. 6,274,150. The third method is the low energy emulsification method. See, e.g., Tadros.

Nanoemulsions offer stability advantages over macroemulsions. Nanoemulsions have dramatically smaller particle sizes and therefore offer higher stability against creaming or sedimentation because their diffusion rate is faster than their sedimentation rate. Also, properly formulated nanoemulsions are more stable against flocculation and coalescence.

Given the benefits of nanoemulsions and the length of time for which nanoemulsions have been known, it is surprising that only a few true nanoemulsions are found in the personal care market today. For example, the PIT method for making nanoemulsions was introduced well over 40 years ago by Shinoda and Arai in 1964. Knowledge of high pressure homogenization methods is even older.

The present inventors believe that there are at least two major reasons why there has not been more widespread use of nanoemulsion technology in personal care. First, nanoemulsions tend to have very low viscosities and poor aesthetics. Nanoemulsions are being used in the medical field for targeted drug delivery, but in that industry, aesthetics are not a concern. Second, many strategies typically used to impart aesthetic properties can also cause nanoemulsion instability. For example, the use of hydrocolloids is a classic formulation strategy to manage a product's rheological behavior, but the choice of hydrocolloids often creates stability challenges for nanoemulsions. The choice and amount of surfactant is another cause of instability.

One important issue when exploring nanoemulsion stability is a phenomenon called Ostwald ripening. Ostwald ripening is caused by a Laplace pressure gradient between drops of different sizes. Laplace's Law can be stated as follows: ΔP=2·T/r. In other words, the pressure inside a drop exceeds the pressure outside the drop by twice the surface tension, divided by the radius. The smaller a drop, the more the pressure inside it exceeds the pressure on the outside. This dynamic can cause smaller drops to fuse, ultimately causing a loss of the nanoemulsion. Stabilizing these forces may become a very time-consuming event for a motivated research team. Literature supports this point of view. See, e.g., Salager et al., “Nanoemulsions: Where Are They Going To?”, Colloidi, Tpoint 2, pp. 12-14 (2003); Izquierdo et al., “Phase Behavior and Nano-Emulsion Formation by the Phase Inversion Temperature Method”, Langmuir, vol. 20, pp. 6594-6598 (2004).

Macroemulsions have known benefits as well. Those include greater rheological control, better “rub-in” qualities and more variable choice of surface conditioning agents. While macroemulsions do not experience the same instability phenomena, i.e., Ostwald ripening, as nanoemulsions, it would be expected that the combination of a macroemulsion with a nanoemulsion would lead to more compounded stability challenges for the resulting product due to Laplace's Law and the high pressure differential between droplets of vastly different diameters.

Accordingly, there is a need to develop an emulsion delivery system which enjoys the purported benefits of nanoemulsion technology, but which does not suffer from the instability issues typically associated therewith. To the inventors' knowledge, no mixed emulsion systems, as set forth in the present invention, exist in the market today.

### SUMMARY OF THE INVENTION

The present invention is directed to a stable mixed emulsion comprising semisolid dispersions of at least two discrete and distinctly different particle size ranges. In a preferred embodiment of the invention, the semisolid dispersions comprise nanoemulsion semisolid dispersions having a particle size range of less than 1 μm and macroemulsion semisolid dispersions having a particle size range of greater than 1 μm. In further preferred embodiments, the nanoemulsion semisolid dispersions have a particle size ranging from 10 nm to 900 nm and the macroemulsion semisolid dispersions have a particle size ranging from 1 μm to 300 μm. In certain preferred embodiments of the invention, a weight ratio of nanoemulsion semisolid dispersions to macroemulsion semisolid dispersions ranges from 20:1 to 1:20.

In certain embodiments of the present invention, the stable mixed emulsion further comprises a continuous phase which contains water and a hydrocolloid. In preferred embodiments, the hydrocolloid is a polyacrylate. In other preferred embodiments, the hydrocolloid is present in an amount ranging from 0.01% to 2% by weight of the stable mixed emulsion. In other preferred embodiments, the water is present in an amount ranging from 20% to 99.9% by weight of the stable mixed emulsion.

In further preferred embodiments of this invention, the nanoemulsion semisolid dispersions comprise at least two nonionic emulsifiers and at least one lipophilic ingredient and/or the macroemulsion semisolid dispersions comprise at least one nonionic emulsifier and at least one lipophilic ingredient. In still more preferred embodiments, the nanoemulsion semisolid dispersions comprise at least two nonionic emulsifiers, wherein at least one of the emulsifiers has an HLB (as defined below) of 8 or more and at least one of the emulsifiers has an HLB below 8 and/or a weight ratio of lipophilic ingredient to emulsifier in the nanoemulsion semisolid dispersions ranges from 1:1 to 20:1. In still further preferred embodiments, the macroemulsion semisolid dispersions comprise at least one emulsifier having an HLB ranging from 8 to 18 and/or a weight ratio of lipophilic ingredient to emulsifier in the macroemulsion semisolid dispersions ranges from 20:1 to 1:3.

The present invention is further directed to a skin care composition comprising the stable mixed emulsion of the present invention.

The present invention is additionally directed to a method of making a stable mixed emulsion comprising the steps of: providing a nanoemulsion; hydrating a hydrocolloid in a separate vessel; adding the nanoemulsion to the hydrated hydrocolloid; mixing macroemulsion oil phase ingredients in another separate vessel to form macroemulsion semisolid dispersions; adding the macroemulsion semisolid dispersions to the hydrated hydrocolloid and the nanoemulsion; and mixing until uniform to prepare the stable mixed emulsion.

In a preferred embodiment of the present invention, the nanoemulsion is prepared using the phase inversion temperature method.

### BRIEF DESCRIPTION OF THE DRAWINGS

FIG. 1 is a particle size analysis of the nanoemulsion portion of the mixed emulsion of Example 1.

FIG. 2 is a particle size analysis of the finished mixed emulsion of Example 1.

### DETAILED DESCRIPTION OF THE INVENTION

As used herein, “stable” refers to the absence of significant change in droplet/particle size distribution or the absence of visible phase separation for a period prior to use which is necessary for storage and/or display; such a degree of stability can be predicted by the absence of significant change in droplet/particle size distribution or the absence of visible phase separation for a period of at least 3 months at 40° C. or a period of at least 1 week at 50° C. As used herein, “nanoemulsion” refers to an emulsion typically having a particle size of less than 1 μm, i.e., a sub-micron emulsion. As used herein, “macroemulsion” refers to an emulsion typically having a particle size of greater than 1 μm. As used herein, “HLB” refers to hydrophilic lipophilic balance (as defined by ICI; see http://www.uniqema.com/press/news8993.htm). As used herein, “semisolid dispersion” refers to the discontinuous phase of an emulsion, i.e., a droplet, a dispersed phase. As used herein, “discrete and distinctly different” refers to two particle sizes that are statistically distinguishable. As used herein, “continuous phase” refers to the phase external to the dispersed discontinuous phase in an emulsion. As used herein, “oil phase” refers to the combination of emulsifier(s) and lipophilic material(s) used to form semisolid dispersions.

The first embodiment of the present invention is directed to a stable mixed emulsion comprising semisolid dispersions of at least two discrete and distinctly different particle size ranges. Nanoemulsions are known in the art; macroemulsions are known as well. The present invention relates to a novel system wherein both nanoemulsion semisolid dispersions and macroemulsion semisolid dispersions are present. Typically, the weight ratio of nanoemulsion semisolid dispersions to macroemulsion semisolid dispersions in the stable mixed emulsion of the present invention ranges from about 20:1 to about 1:20, preferably from about 6:1 to about 1:6, and more preferably from about 1:1 to about 4:1.

According to the present invention, nanoemulsion semisolid dispersions typically have a particle size of less than 1 μm, preferably between about 10 nm and about 900 nm, and more preferably between about 100 nm and about 400 nm.

For purposes of the present invention, nanoemulsion semisolid dispersions comprise at least two nonionic emulsifiers and at least one lipophilic ingredient. Importantly, the weight ratio of lipophilic ingredient to emulsifier in the nanoemulsion semisolid dispersions ranges from about 1:1 to about 20:1, preferably from about 2:1 to about 10:1, and more preferably from about 3:1 to about 6:1. The inventors currently believe that the weight ratio of lipophilic ingredient to emulsifier in the nanoemulsion semisolid dispersions is key to achieving stability for the mixed emulsion system.

Also according to the present invention, macroemulsion semisolid dispersions typically have a particle size of greater than 1 μm, preferably between about 1 μm and about 300 μm, and more preferably between about 15 μm and about 90 μm.

For purposes of the present invention, macroemulsion semisolid dispersions comprise at least one nonionic emulsifier and at least one lipophilic ingredient. Importantly, the weight ratio of lipophilic ingredient to emulsifier in the macroemulsion semisolid dispersions ranges from about 20:1 to about 1:3, preferably from about 10:1 to about 1:1, and more preferably from about 6:1 to about 2:1. The inventors currently believe that the weight ratio of lipophilic ingredient to emulsifier in the macroemulsion semisolid dispersions is also key to achieving stability for the mixed emulsion system.

In a preferred embodiment of this invention, the nanoemulsion semisolid dispersions employ at least two nonionic emulsifiers, at least one of the emulsifiers having a high HLB of 8 or more, preferably ranging from about 14 to about 15, and at least one of the emulsifiers having a low HLB of below 8, preferably about 4. In another preferred embodiment of this invention, the macroemulsion semisolid dispersions employ at least one nonionic emulsifier having an HLB ranging from about 8 to about 18. It is worthwhile to note that, because the emulsifier(s) employed for the nanoemulsion semisolid dispersions and for the macroemulsion semisolid dispersions can be different materials, all emulsifiers employed must be compatible, i.e., have similar solubility properties, etc. in order to preserve stability. The inventors currently believe that the choice of emulsifiers is another key to achieving stability for the mixed emulsion system. Thus, not only is the weight ratio of lipophilic ingredient to emulsifier in each of the nanoemulsion semisolid dispersions and the macroemulsion semisolid dispersions key, but also emulsifier selection is key.

Nonionic emulsifiers suitable for use in the present invention are quite diverse; they are limited only by their ability to satisfy the above-noted HLB requirements. Suitable nonionic emulsifiers or surfactants can be found in Surfactants in Cosmetics, 2d edition, M. Rieger et al., eds., Marcel Dekker, Inc., New York, pp. 19-28 (1997) and in Harry's Cosmeticology, 8^{th} edition, M. Rieger, ed., Chemical Publishing Co., Inc., New York, pp. 202-209 (1997). Generally, nonionic surfactants are substances in which the molecule carries no charge. The hydrophobe can be highly variable, but the hydrophilic head generally includes a polyether group or at least one —OH group.

The nonionic surfactants most useful for purposes of the present invention can be conveniently divided into three large groups. The first of these groups is alcohols [R—CH₂—OH], for example, cetearyl alcohol; preferably the alkyl R group has a chain length ranging from 6-22 carbons.

The second of these groups is esters. Esters include glycerides such as glyceryl stearate and glyceryl oleate; ethoxylated glycerides such as PEG-20 glyceryl stearate; polyglyceryl esters such as polyglyceryl-2-caprate; sorbitan esters such as Tween 80 and sorbitan oleate; carbohydrate esters such as sucrose distearate and PEG-120 methyl glucose dioleate; ethoxylated carboxylic acids such as ethoxylated fatty acids like PEG-150 oleate and PEG-6 dilaurate; and phosphoric acid triesters such as trideceth-3 phosphate. Further, a large number of nonionic esters can be prepared by reaction of alcohols or polyalcohols with a variety of natural and or hydrogenated oils, i.e., via alcohol-oil transesterification. Most commonly, the oils used are castor oil or hydrogenated castor oil, or an edible vegetable oil such as corn oil. Preferred alcohols include glyceryol, propylene glycol, ethylene glycol, polyethylene glycol, sorbitol and pentaerythritol. Examples of transesterified nonionic surfactants include, without limitation, PEG-40 hydrogenated castor oil, PEG-60 corn glycerides, and PEG-40 palm kernel oil.

The third of these groups is ethers. Ethers include ethoxylated alcohols such as laureth 4, ceteareth-10 and ceteareth-20; ethoxylated (propoxylated) polysiloxanes such as dimethicone copolyols and PEG/PPG-15/15 dimethicone; ethoxylated polypropylene oxide ethers such as poloxamer 407, PPG-9 buteth-12; and alkyl glycosides such as decyl glucoside.

Exact carbon chain lengths and the degree of propoxylation/ethoxylation for all of the above-noted surfactants will determine the HLB value of nonionic emulsifiers such as these. Ultimately it is important to note that any nonionic emulsifier can be used for purposes of this invention as long as it satisfies the HLB requirements set forth above for nanoemulsion and macroemulsion semisolid dispersions. In certain preferred embodiments of this invention, PEG 40 hydrogenated castor oil, ceteareth-20 and glyceryl oleate are preferred for use in the nanoemulsion semisolid dispersions; in certain preferred embodiments, PEG 40 hydrogenated castor oil and laureth-4 are preferred for use in the macroemulsion semisolid dispersions.

Lipophilic ingredients suitable for use in either or both of the nanoemulsion and macroemulsion semisolid dispersions include, without limitation, aliphatic hydrocarbons (straight or branched chain) such as mineral oil and isododecane; natural oils such as soybean oil, sunflower seed oil, olive oil, palm oil, wheat germ oil, shark liver oil, squalene, shea butter; esters such as isopropyl myristate; branched chained esters, e.g., chain length from 3-30, such as cetyl ethylhexanoate; waxes such as beeswax, jojoba wax, and carnuba wax; silicones; active ingredients; and mixtures thereof.

Preferred for use in the macroemulsion semisolid dispersions are silicones, more specifically, organopolysiloxanes selected from polyalkylsiloxanes, alkyl substituted dimethicones, cyclomethicones, trimethylsiloxysilicates, dimethiconols, polyalkylaryl siloxanes, dimethicone crosspolymers, and mixtures thereof. More preferred for use in the macroemulsion semisolid dispersions are dimethicone crosspolymers, polydimethylsiloxanes and cyclomethicones. Preferred for use in the nanoemulsion semisolid dispersions are branched chain esters such as cetyl ethylhexanoate.

Lipophilic active ingredients as noted above may include actives such as anti-inflammatory agents, both steroidal (hydrocortisone, beclomethasone, etc.) and non-steroidal (oxicams, salicylates, acetic acid derivatives, fenamates, propionic acid derivatives, pyrazoles) as well as natural anti-inflammatory agents (aloe vera, bisabolol, etc.); antioxidants (ursoic acid, tocopherol, etc.); oil soluble vitamins (D, A, folic acid, etc.); topical anesthetics (benzocaine, lidocaine, etc.); antimicrobial agents; antifungal agents; sunscreen agents (physical blockers such as metallic oxides like titanium and zinc oxides; and UVA & UVB absorbers such as octyl methoxycinnamate, avobenzone, 4-methylbenzylidene camphor); skin-lightening agents; anti-acne agents (salicylic acid, benzoyl peroxide, azelaic acid, isotretinoin, etc.); antibiotics (clindamycin, erythromycin, metronidazol, sulfacetamide, etc.) and combinations thereof. U.S. Pat. No. 6,492,326, the disclosure of which is incorporated by reference herein, sets forth a useful discussion with regard to both lipophilic active ingredients and silicones which may advantageously be employed for use in the present invention.

The lipophilic ingredient(s) used in the nanoemulsion semisolid dispersions can be the same as or different from the lipophilic ingredient(s) used in the macroemulsion semisolid dispersions.

Also present in the stable mixed emulsion of the first embodiment is a continuous phase typically comprising water and a hydrocolloid. Water is preferably employed in an amount ranging from 20% to 99.9%, more preferably from about 40% to about 98%, and most preferably from about 60% to about 95%, by weight of the stable mixed emulsion.

The hydrocolloid is preferably employed in an amount ranging from 0.01% to 2%, more preferably from 0.1 to 1.2%, and most preferably from 0.2% to 0.8% by weight of the stable mixed emulsion. The hydrocolloid used in the present invention is preferably a polyacrylate. U.S. Patent Application Publication No. 2004/0202635, the entire disclosure of which is incorporated by reference herein, contains an effective description of such hydrocolloids suitable for use in this invention. In particular, acrylate copolymers and/or acrylate-alkyl acrylate copolymers which are available under the trade names Carbopol® 1382, Carbopol® 981, Carbopol® 5984, Aqua SF-1 (NOVEON Inc.), and Aculyn® 33 (International Specialty Products Corp.). Also useful as the hydrocolloid of the present invention are copolymers of C₁₀₋₃₀-alkyl acrylates and one or more monomers of acrylic acid, of methacrylic acid or esters thereof which are crosslinked with an allyl ether of sucrose or an allyl ether of pentaerythritol. Additionally, compounds which carry the INCI name “acrylates/C₁₀₋₃₀ alkyl acrylate crosspolymer” are advantageous. Particularly advantageous are those polymers available under the trade names Pemulen TR1 and Pemulen TR2 from NOVEON Inc., Ultrez 21 and Carbopol® ETD 2020. What is more, compounds which carry the INCI name “acrylates/C₁₂₋₂₄ pareth-25 acrylate copolymer” (obtainable under the trade names Synthalen® W2000 from 3V Inc.), the INCI name “acrylates/steareth-20 methacrylate copolymer” (obtainable under the trade names Aculyn® 22 from International Specialty Products Corp.), the INCI name “acrylates/steareth-20 itaconate copolymer” (obtainable under the trade names Structure 2001® from National Starch), the INCI name “acrylates/aminoacrylates/C₁₀₋₃₀ alkyl PEG-20 itaconate copolymer” (obtainable under the trade names Structure Plus® from National Starch) and similar polymers are also useful for purposes of the present invention. The hydrocolloids preferred for use in the present invention include Carbopol® ETD2020 and Ultrez 21.

Other ingredients suitable for use in the stable mixed emulsion of the present invention include, without limitation, humectants, hydrocolloid/rheology modifiers, actives, color, fragrance, preservatives, antioxidants, chelators, aqueous actives, anionic hydrocolloid neutralizers (such as triethanolamine and sodium hydroxide), water soluble natural extracts, water soluble active ingredients, water soluble vitamins, and combinations thereof. One of ordinary skill in the art would readily be able to determine the amount of these other ingredients suitable for use in the stable mixed emulsion of the present invention based on the desired end product. In addition, one of ordinary skill in the art would readily appreciate that such other ingredients may be present in the semisolid dispersions or in the continuous phase of the stable mixed emulsion as appropriate.

The mixed emulsions of the present invention are stable. In other words, there is minimal flocculation, Ostwald's ripening (of the nanoemulsion portion), or coalescence of semisolid dispersions, etc., for a period prior to use which is necessary for storage and/or display, where such a degree of stability can be predicted by the absence of significant change in droplet/particle size distribution or the absence of visible phase separation for a period of at least 3 months at 40° C. or a period of at least 1 week at 50° C. In addition to stability, the mixed emulsions of the present invention exhibit significant improvement in skin feel, as compared to a straight nanoemulsion or to a straight macroemulsion. A nanoemulsion typically absorbs quickly leaving minimal after-feel, while a macroemulsion with the same lipophilic ingredients tends to absorb more slowly but can leave a more elegant skin feel; combination of the two results in positive enhancement. In addition, the mixed emulsion system allows for rapid skin penetration of active materials, enhanced delivery and a more uniform deposition of active materials on the skin. What is more, the mixed emulsion of the present invention enjoys the benefits of nanoemulsion systems as set forth by Tadros et al.

A second embodiment of the present invention is directed to a skin care composition comprising a stable mixed emulsion in accordance with the first embodiment of this invention. For purposes of this invention, “skin care composition” refers to a topical composition which can be applied to any or all of skin, mucous membranes, hair, etc., for any purpose. The skin care composition may additionally comprise other typical ingredients. Other typical ingredients include, without limitation, humectants, preservatives, fragrance, color, natural extracts, and combinations thereof. The stable mixed emulsion of the first embodiment of the invention is typically employed in an amount ranging from 99.99% to 1.0% by weight of a skin care composition of the second embodiment.

The third embodiment of the present invention is directed to a method of making a stable mixed emulsion. In the first step of this method, a nanoemulsion is provided. The nanoemulsion can be obtained from any known source (so long as it satisfies the characteristics outlined above with regard to the ratio of lipophilic material to emulsifier). Alternatively, the nanoemulsion can be prepared according to any known nanoemulsion technique; such techniques include, without limitation, phase inversion temperature (PIT), high pressure homogenization, low energy oil/water inversion method.

In a preferred embodiment of this invention, the nanoemulsion is prepared via a PIT method and the step of providing the nanoemulsion comprises steps such as heating water to a temperature greater than a phase inversion temperature of an emulsifier used in the nanoemulsion; heating an oil phase of the nanoemulsion in a separate vessel to the same temperature as the water, wherein the oil phase comprises at least the emulsifier and a lipophilic material; adding the heated oil phase to the heated water to obtain a mixture; and cooling the mixture to a temperature below the phase inversion temperature of the emulsifier to form the nanoemulsion.

In the second step of the method of the third embodiment, a hydrocolloid is hydrated in a separate vessel. Hydrocolloid hydration is well within the skill of one of ordinary skill in this art and is typically accomplished by mixing the hydrocolloid with a suitable amount of water, accompanied by agitation and heating. At this point in the inventive method, other ingredients as set forth above with regard to the first embodiment of the invention may optionally be added if so desired.

In the next step of the method of the third embodiment, the nanoemulsion is added to the hydrated hydrocolloid in any suitable manner. Typically this is accomplished at room temperature with mixing. In the fourth step of the method of the third embodiment, macroemulsion oil phase ingredients are mixed in another separate vessel to form macroemulsion semisolid dispersions. In the final steps of the method of the third embodiment of the invention, the macroemulsion semisolid dispersions are added to the hydrated hydrocolloid and the nanoemulsion and mixed until uniform to prepare the stable mixed emulsion.

All of the details regarding the third embodiment of the present invention, i.e., ratio of nanoemulsion semisolid dispersions to macroemulsion semisolid dispersions, types of emulsifiers, lipophilic materials, ratios of lipophilic material to emulsifier, type and amount of water and hydrocolloid, etc., are the same as those regarding the first embodiment of the invention set forth above.

Specific embodiments of the invention will now be demonstrated by reference to the following examples. It should be understood that these examples are disclosed solely by way of illustrating the invention and should not be taken in a way to limit the scope of the present invention.

### EXAMPLE 1

A mixed emulsion was made to contain the components as set forth in Table 1 below.

TABLE 1

| Ingredients | % w/w |
|---|---|
| A. nanoemulsion phase | |
| PEG-40 hydrogenated castor oil | 0.4 |
| ceteareth-20 | 0.3 |
| glyceryl oleate | 0.3 |
| cetyl octanoate | 4.0 |
| water | 20.0 |
| B. water phase | |
| water | q.s. a.d. |
| glycerin | 3.0 |
| Carbopol ETD 2020 | 0.2 |
| neutralizer | q.s. |
| preservatives | q.s. |
| color | q.s. |
| C. macroemulsion phase | |
| Jeesperse HD (dimethicone | 6.0 |
| crosspolymer-3, isododecane, | |
| laureth-4) | |
| PEG-40 hydrogenated castor oil | 2.0 |
| fragrance | q.s. |

*q.s.a.d. to 100%

The nanoemulsion phase ingredients are heated to a temperature about 5° C. above the phase inversion temperature with the use of a conductance meter. In this example, the phase inversion temperature is about 75° C.-80° C., so the nanoemulsion phase ingredients were mixed at 85° C. Then the mixture was cooled with turbine mixing slowly through the phase inversion temperature and then rapidly to room temperature. Next, the hydrocolloid, Carbopol ETD 2020, was fully hydrated in water and then the remaining water phase ingredients were added. Then, the nanoemulsion phase was added to the water phase using turbine agitation. In a separate container, the macroemulsion phase ingredients were mixed together. It should be noted that if the macroemulsion employs ingredients with melting points above room temperature, the macroemulsion phase ingredients, as well as the vessel containing the nanoemulsion and water phases, should be heated to a temperature sufficient to melt the macroemulsion ingredients but not above the phase inversion temperature. Finally, the macroemulsion phase was added to the already combined nanoemulsion and water phases with slow turbine mixing. All phases were mixed until a uniform mixed emulsion system was achieved.

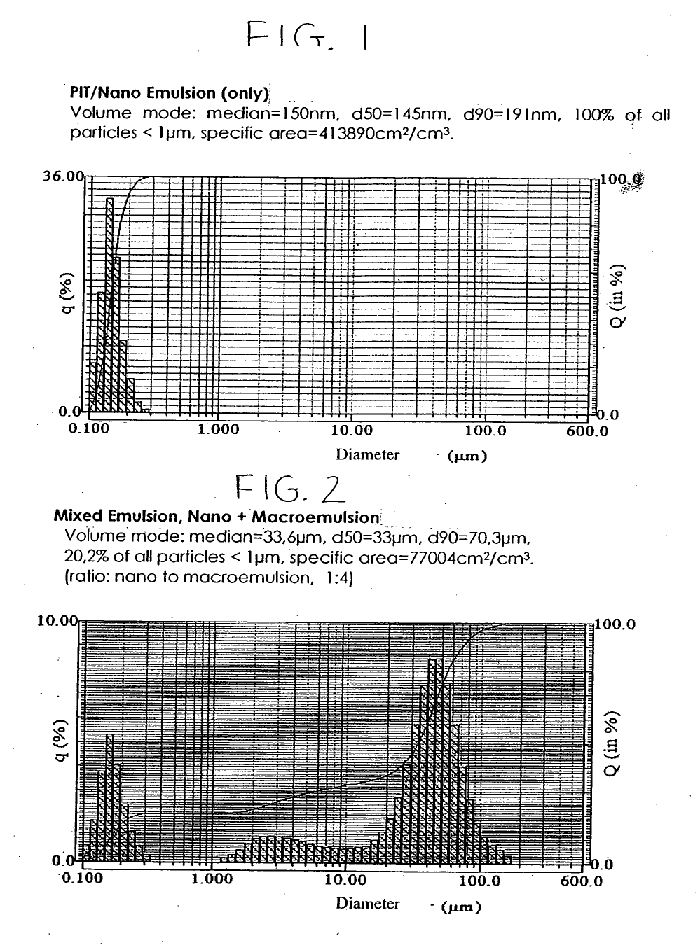

Particle size analysis was undertaken at two stages of this example. First, particle size analysis was conducted for the nanoemulsion phase only (FIG. 1); then, particle size analysis was conducted for the finished mixed emulsion (FIG. 2). Analysis was completed in conformity with standard testing parameters using a Horiba LA 300 with a measurement range of 0.1 to 600 microns.

### EXAMPLES 2-6

Mixed emulsions were made to contain the components as set forth in Table 2 below using the basic procedure outlined in Example 1.

TABLE 2

| | Ex. 2 | Ex. 3 | Ex. 4 | Ex. 5 | Ex. 6 |
|---|---|---|---|---|---|
| Ingredients | % w/w | % w/w | % w/w | % w/w | % w/w |
| A. nanoemulsion phase | | | | | |
| PEG-40 hydrogenated | 1.2 | 1.0 | 0.4 | 0.4 | 0.4 |
| castor oil | | | | | |
| ceteareth-20 | 0.9 | 0.7 | 0.3 | 0.3 | 0.3 |
| glyceryl stearate | 0.9 | 0.6 | 0.3 | 0.3 | 0.3 |
| cetyl octanoate | — | 5.0 | 4.0 | 4.0 | 4.0 |
| soybean oil | 8.0 | — | — | — | — |
| shea butter | 2.0 | — | — | — | — |
| hydrocortisone acetate | — | 1.0 | — | — | — |
| tocopherol | — | — | 0.5 | — | — |
| water | 50.0 | 50.0 | 20.0 | 20.0 | 20.0 |
| B. water phase | | | | | |
| water | q.s.a.d. | q.s.a.d. | q.s.a.d. | q.s.a.d. | q.s.a.d. |
| glycerin | — | 3.0 | 3.0 | 3.0 | 3.0 |
| acrylates/C₁₀₋₃₀ alkyl | 0.2 | 0.2 | 0.2 | — | 0.2 |
| acrylate crosspolymer | | | | | |
| acrylates/steareth-20 | — | — | — | 1.0 | — |
| methacrylate | | | | | |
| copolymer | | | | | |
| neutralizer | qs | qs | qs | qs | qs |
| preservatives | qs | qs | qs | qs | qs |
| color | qs | qs | qs | qs | qs |
| C. macroemulsion phase | | | | | |
| Jeesperse HD | — | 6.0 | 6.0 | 6.0 | — |
| (dimethicone | | | | | |
| crosspolymer-3, | | | | | |
| isododecan, laureth-4) | | | | | |
| DC 9546 silicone | — | — | — | — | 2.0 |
| polyglyceryl-3 | — | — | — | — | 1.0 |
| disiloxane | | | | | |
| dimethicone | | | | | |
| octyldodecyl | 5.0 | — | — | — | — |
| myristate | | | | | |
| PEG-40 hydrogenated | 2.0 | 2.0 | 2.0 | 2.0 | 2.0 |
| castor oil | | | | | |
| fragrance | qs | qs | qs | qs | qs |

*q.s.a.d. to 100%

### Stability Testing

The mixed emulsion system of Example 1 was tested for stability according to International Conference on Harmonization (ICH) guidelines. More particularly, the mixed emulsion system was tested for a period of at least 3 months at 40° C. and for a period of at least 1 week at 50° C. The tested mixed emulsion system exhibited no physical separation of water and oil.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A stable mixed emulsion comprising semisolid dispersions of at least two discrete and distinctly different particle size ranges.

2. The stable mixed emulsion of claim 1, wherein the semisolid dispersions comprise nanoemulsion semisolid dispersions having a particle size range of less than 1 μm and macroemulsion semisolid dispersions having a particle size range of greater than 1 μm.

3. The stable mixed emulsion of claim 2, wherein the nanoemulsion semisolid dispersions have a particle size ranging from 10 nm to 900 nm.

4. The stable mixed emulsion of claim 2, wherein the macroemulsion semisolid dispersions have a particle size ranging from 1 μm to 300 μm.

5. The stable mixed emulsion of claim 2, wherein a weight ratio of nanoemulsion semisolid dispersions to macroemulsion semisolid dispersions ranges from 20:1 to 1:20.

6. The stable mixed emulsion of claim 1 further comprising a continuous phase, said continuous phase comprising water and a hydrocolloid.

7. The stable mixed emulsion of claim 6, wherein the hydrocolloid is a polyacrylate.

8. The stable mixed emulsion of claim 6, wherein the hydrocolloid is present in an amount ranging from 0.01% to 2% by weight of the stable mixed emulsion.

9. The stable mixed emulsion of claim 6, wherein the water is present in an amount ranging from 20% to 99.9% by weight of the stable mixed emulsion.

10. The stable mixed emulsion of claim 2, wherein the nanoemulsion semisolid dispersions comprise at least two nonionic emulsifiers and at least one lipophilic ingredient.

11. The stable mixed emulsion of claim 10, wherein at least one of the at least two emulsifiers has an HLB of 8 or more and at least one of the at least two emulsifiers has an HLB below 8.

12. The stable mixed emulsion of claim 10, wherein a weight ratio of lipophilic ingredient to emulsifier in the nanoemulsion semisolid dispersions ranges from 1:1 to 20:1.

13. The stable mixed emulsion of claim 2, wherein the macroemulsion semisolid dispersions comprise at least one nonionic emulsifier and at least one lipophilic ingredient.

14. The stable mixed emulsion of claim 13, wherein the at least one emulsifier has an HLB ranging from 8 to 18.

15. The stable mixed emulsion of claim 13, wherein a weight ratio of lipophilic ingredient to emulsifier in the macroemulsion semisolid dispersions ranges from 20:1 to 1:3.

16. The stable mixed emulsion of claim 10 or claim 13, wherein the at least one lipophilic ingredient is selected from the group consisting of aliphatic hydrocarbons, natural oils, squalene, esters, branched chained esters, volatile hydrocarbons, waxes, silicones, active ingredients and combinations thereof.

17. The stable mixed emulsion of claim 1, wherein the stable mixed emulsion is stable for at least a period of three years at 22° C.

18. A skin care composition comprising the stable mixed emulsion of claim 1.

19. The skin care composition of claim 18 further comprising ingredients selected from the group consisting of humectants, preservatives, fragrance, color, natural extracts, and combinations thereof.

20. A method of making a stable mixed emulsion comprising the steps of:
providing a nanoemulsion;
hydrating a hydrocolloid in a separate vessel;
adding the nanoemulsion to the hydrated hydrocolloid;
mixing macroemulsion oil phase ingredients in another separate vessel to form macroemulsion semisolid dispersions;
adding the macroemulsion semisolid dispersions to the hydrated hydrocolloid and the nanoemulsion; and
mixing until uniform to prepare the stable mixed emulsion.

21. The method of making a stable mixed emulsion according to claim 20, wherein the step of preparing the nanoemulsion comprises a phase inversion temperature method.

22. The method of making a stable mixed emulsion according to claim 20, wherein the weight ratio of nanoemulsion semisolid dispersions to macroemulsion semisolid dispersions ranges from 20:1 to 1:20.

23. The method of making a stable mixed emulsion according to claim 20, wherein the ratio of a lipophilic ingredient to emulsifier in the nanoemulsion semisolid dispersions ranges from 1:1 to 20:1.

24. The method of making a stable mixed emulsion according to claim 20, wherein the particle size of the nanoemulsion semisolid dispersions is less than 1 μm.

25. The method of making a stable mixed emulsion according to claim 24, wherein the particle size of the nanoemulsion semisolid dispersions ranges from 10 nm to 900 nm.

26. The method of making a stable mixed emulsion according to claim 20, wherein the ratio of a lipophilic material to emulsifier in the macroemulsion semisolid dispersions ranges from 20:1 to 1:3.

27. The method of making a stable mixed emulsion according to claim 20, wherein the particle size of the macroemulsion semisolid dispersions is greater than 1 μm.

28. The method of making a stable mixed emulsion according to claim 27, wherein the particle size of the macroemulsion semisolid dispersions ranges from 1 μm to 300 μm.
